(19) United States
(12) Patent Application Publication
(10) Pub. No.: US 2005/0027005 A1
(43) Pub. Date: Feb. 3, 2005

(54) Nutrient compositions and methods for sustenance and promotion of positive metabolic energy levels in a targeted manner

(76) Inventor: Matthias Boldt, Oxnard, CA (US)

Correspondence Address: JOSEPH E. CHOVANES, SUITE 329, 5 GREAT VALLEY PARKWAY, MALVERN, PA, 19355, US

(21) Appl. No.: 10/633,232

(22) Filed: Aug. 2, 2003

(51) Int. Cl. A61K 31/205; A61K 31/198

(52) U.S. Cl. 514/561; 514/554

(57) ABSTRACT

Nutrient compositions and methods that sustain and promote positive metabolic energy levels in a targeted manner are disclosed. Methods utilize endogenous energy stores (fat oxidation), increase use of those stores (increasing transport rate), increase available energy (increasing the ability to perform ADP to ATP phosphorylation,) as well as decrease catabolism and increase protein synthesis. Compositions are also disclosed, and include Mono- or Dicreatine-HMB salt; Putrescine Dihydrochloride; Alanine; L-Glutamine, which may be combined with Alanine in a 1:2 to 2:1 molecular ratio; Trimethylglycine; and Guanidinopropionic Acid.

### FIELD OF THE INVENTION

The present invention relates to compositions and methods that sustain and promote metabolic energy levels. More particularly, the present invention relates to nutrient compositions and methods that sustain and promote positive metabolic energy levels in a targeted manner.

### BACKGROUND OF THE INVENTION

Negative energy balance, or energy intake below body maintenance needs, can occur as a result of a number of conditions. For example, conditions such as physiological stress, restricted caloric intake (dieting, etc.), strenuous physical exercise, physical trauma, bum injury, malnutrition, maldigestion, chemotherapy, anorexia, etc., can cause a negative energy balance. (Energy, as used herein, includes various calories as well as nutrients, e.g., amino acids, natural growth factors, methyl groups, etc.)

A negative energy balance may affect the body in various ways. For example, the body's energy stores, e.g. fat, glycogen, muscle, etc. may be used for energy if the body has a negative energy balance. An individual may attempt to use a negative energy balance to his or her benefit, e.g. by dieting and attempting to increase his or her proportion of lean body mass to fat may try to impose a negative energy balance on his or her body—eating less calories in an attempt to have the body oxidize (“bum”) fat for energy. However, often attempts to impose a negative energy balance are misguided. For example, merely eating less calories will consume both body fat and muscle stores and result in little if any change in overall lean body mass/body fat percentages. Thus, methods to use negative energy balances in the body for desired change in the body may have less than desirable effects.

A positive energy balance, or energy intake above body maintenance needs, may create difficulties as well. By ingesting too much energy, the body may store the energy in the form of fat, thus leading to obesity. Yet a positive energy balance may be helpful and even necessary for body repair and/or growth. For example, a burn victim may need energy intake far above what is necessary for maintenance in order to repair tissue. For example, an elite athlete may need energy intake far above what is necessary for maintenance in order to grow new muscle tissue.

Balancing a body's needs and energy balance, therefore, may be difficult. Moreover, where vigorous exercise is undertaken, an appropriate energy balance may be extremely difficult to achieve. For example, vigorous muscle exercise causes damage to the muscle tissue and its cells. When the muscle cells repair the damage, they grow: they may increase their size (e.g., protein growth), they may increase their cellular components and perhaps increase their number: all in order to resist future damage. Further exercise, if effective, damages the repaired cells in order to further promote growth, subsequent repair, etc. Each stage of this exercise-damage-repair-growth cycle requires energy. Yet any energy supplied to the body for the muscle exercise-damage-repair-growth cycle should be targeted so as not to lead to undesired consequences (e.g., fat tissue accumulation.)

Accordingly, it would be beneficial to provide nutrient compositions and methods that maintain a positive metabolic energy balance in a targeted manner.

It would be further beneficial to provide nutrient compositions and methods that maintain a positive metabolic energy balance in a targeted manner through limited excess calories.

### SUMMARY OF THE INVENTION

The present invention relates to compositions and methods that sustain and promote metabolic energy levels. More particularly, the present invention relates to nutrient compositions and methods that sustain and promote positive metabolic energy levels.

A composition of a preferred embodiment comprises: Mono- or DiCreatine-HMB Salt, Putrescine Dihydrochloride, Amino Acid dipeptides selected from the group of L-Alanine and L-Glutamine, Trimethylglycine, and Guanidinopropionic Acid. A method of a preferred embodiment comprises administration of a compound comprising Mono- or DiCreatine-HMB, Putrescine Dihydrochloride, Amino Acid dipeptides selected from the group consisting of L-Alanine and L-Glutamine, Trimethylglycine, and Guanidinopropionic Acid.

An Alanine dipeptide that is chemically bound at a 1:1, 2:1 or 1:2 molecular ratio to L-Glutamine is used in some especially preferred embodiments.

In yet other embodiments, Creatine and HMB may be administered in non-compound form.

### DETAILED DESCRIPTION OF THE PREFERRED EMBODIMENTS

The present invention relates to compositions and methods that sustain and promote metabolic energy levels. More particularly, the present invention relates to nutrient compositions and methods that sustain and promote positive metabolic energy levels in a targeted manner.

Embodiments provide compositions and methods that sustain and promote metabolic energy levels through targeting specific body tissues. That is, they attempt to provide energy to skeletal muscle so as to make possible an increase in the rate and intensity of muscle use. Because vigorous muscle exercise causes damage to the muscle tissue and its cells, the body repairs the cells after damage. When the muscle cells are repaired, they grow: their size may be increased (e.g., protein synthesis) and the number of cellular components per cell may be increased in order to resist future damage. Further exercise, if effective, must damage the repaired cells in order to further promote growth, subsequent repair, etc.

Since each stage of this exercise-damage-repair-growth cycle requires energy, the greater the muscle development, the greater the energy requirements for the cycle. Thus, embodiments attempt to provide useful energy to the muscle for the cycle. For example, if energy can be supplied so as to allow for increased exercise, then increased damage will result and so growth. (Growth as used herein is intended to include: hypertrophy or physical size of a muscle fiber, as occurs with Type Ia and IIb (also known as fast twitch oxidative and fast twitch glycolytic) muscle fibers; intracellular density, such as occurs with Type I fibers (also known as slow twitch oxidative); as well as hyperplasia, which appears to occur with various types of fibers through various mechanism, e.g. fiber splitting, satellite cell adaptation, stem cell adaptation, etc.)

Embodiments also attempt to inhibit the body's use of its own muscle tissue for energy (muscle catabolism), as well as increasing the rate at which protein synthesis (muscle repair) occurs.

As described above, energy is provided in various embodiments to muscle. Providing energy comprises both direct and indirect methods:
energy is directly provided via amino acids which may assist in fat oxidation and so provide glucose for energy, e.g., Beta-hydroxy Beta-methylbutyrate (HMB);
energy is directly provided via amino acids which may assist in gluconeogenesis and so provide glucose for energy, e.g., L-Alanine;
energy is indirectly provided via amino acids that are used to convert existing energy stores from potential energy to actual energy, e.g., creatine, used to convert ADP (adenosine diphosphate) to ATP (adenosine triphosphate), which in turn, is used by the muscle to contract.

Moreover, in various preferred embodiments, the rate at which energy is provided is increased, that is, via compounds that appear to assist transport of glucose, glycogen and amino acids into the cell, e.g., Guanidinopropionic acid (GPA.)

As was described above, embodiments also attempt to inhibit the body's use of its own muscle tissue for energy (muscle catabolism), as well as increasing the rate at which protein synthesis (muscle repair) occurs. Anticatabolic effects appear to be provided through Glutamine and/or Alanine. Increased synthesis effects appear to be provided through a nutrient-rich environment for cellular growth (e.g. Putrescine Dihydrochloride, Trimethylglycine (TMG), Glutamine and Alanine.)

Thus, the preferred embodiments provide methods to utilize endogenous energy stores (fat oxidation), and to increase use of those stores (increasing transport rate), methods to increase available energy (increasing the ability to perform ADP to ATP phosphorylation,) as well as anticatabolic methods and methods that increase protein synthesis.

The following description of those compounds used in methods of the preferred embodiments may be helpful. It should be understood that embodiments allow for the substitution of any of the compounds with a pharmaceutical and biochemical equivalent or complement, as long as that substitute functions in the same manner as the compound which it replaces in the composition. Compounds used are optimally at least 75% pure.

### Creatine

Creatine as used herein is intended to encompass creatine in any of its various forms, including salts, derivatives and mixtures, e.g. creatine monohydrate, dicreatine malate, etc. In especially preferred embodiments, creatine is combined with HMB in Mono- or Dicreatine-HMB salt.

The amount of creatine in the compositions of the various preferred embodiments will typically be from about 50 mg to about 20 grams daily. It should be noted that this may be, in certain embodiments, administered in various doses throughout the day. In some embodiments, loading may be desired, and therefore the dose of creatine and any other compounds would begin at a higher volume and decrease accordingly.

Creatine appears to provide energy to the body by facilitating conversion of ADP to ATP which is a primary energy molecule for the body. In Type IIb muscle fibers, for example, which are those utilized for high strength, short duration movements, ATP provides the energy that allows the muscles to contract. Once used, ATP is converted to ADP, and is no longer available to fuel the muscle. Creatine permits the conversion of ADP back to ATP.

(Creatine may also have other benefits, e.g., through improving muscle volumization which may be an additional stimulus for protein synthesis.)

### HMB

HMB is a metabolite of the BCAA amino acid Leucine. In especially preferred embodiments, creatine may be combined with HMB in Mono- or Dicreatine-HMB salt.

The amount of HMB in the compositions of the various preferred embodiments will typically be from about 10 mg to about 50 grams daily. It should be noted that this may be, in certain embodiments, administered in various doses throughout the day. The amount of Dicreatine-HMB salt will typically be from 100 mg to 50 grams daily.

HMB appears to reduce muscle catabolism and increase fat burning. Thus, muscle is not broken down as would otherwise be the case and resources that otherwise would be devoted to muscle catabolism may be used instead for muscle metabolism (e.g. muscle growth.)

### Putrescine Dihydrochloride

Putrescine Dihydrochloride (Putrescine) is a polyamine (a diamine), used for cell growth and differentiation. Endogenous biosynthesis of Putrescine may be less than necessary in the exercising individual—thus the body may not be able to grow (and perhaps create) muscle cells as optimally as desired. Exogenous supplementation provides elevated levels of Putrescine that may be necessary for optimum growth and repair.

The amount of Putrescine in the compositions of the various preferred embodiments will typically be from about 10 mg to about 1000 mg daily. It should be noted that this may be, in certain embodiments, administered in various doses throughout the day.

L-Alanine

L-Alanine is an amino acid that assists in producing glucose from glycogen (gluconeogenesis) stored in the liver. Glucose, of course, is then used in a glycolytic reaction by muscles and other tissues for energy. Exogenous supplementation of L-Alanine may increase the rate of gluconeogenesis and thus increase circulating glucose. L-Alanine may also increase the rate of protein synthesis (perhaps by affecting a positive nitrogen balance.) Moreover, as described further below, L-Alanine may have synergistic effects when combined with L-Glutamine.

The amount of L-Alanine in the compositions of the various preferred embodiments will typically be from about 10 mg to about 30 grams daily. It should be noted that this may be, in certain embodiments, administered in various doses throughout the day.

### L-Glutamine

L-Glutamine appears to assist in protein synthesis (perhaps by affecting a positive nitrogen balance,) assist in muscle anticatabolic reactions, and increase muscle volume. Thus L-Glutamine appears to assist in increasing muscle endurance and growth, as well as decreasing muscle catabolism.

L-Glutamine may also have a synergistic effect when combined with Alanine, as Alanine and L-Glutamine are released from the muscle upon exercise. Endogenous supplements may decrease catabolisis of proteins for energy in muscle, as they would not be required to form either Glutamine or Alanine. Thus those Amino Acids that might otherwise be catabolised for energy may be used for synthesis instead. Therefore, in especially preferred embodiments, an Alanine dipeptide is chemically bound at a 1:1, 2:1 or 1:2 molecular ratio to L-Glutamine.

The amount of L-Glutamine in the compositions of the various preferred embodiments will typically be from about 500 mg to 50 grams daily. It should be noted that this may be, in certain embodiments, administered in various doses throughout the day.

The amount of Alanine-Glutamine compound will typically be from about 10 mg to about 50 grams daily. It should be noted that this may be, in certain embodiments, administered in various doses throughout the day.

### GPA

GPA appears to increase insulin sensitivity and the rate of fat oxidation. Moreover, GPA may act, alone and with insulin, to promote glucose and insulin utilization. This latter attribute may be especially helpful where protein deficiency and/or caloric malnutrition conditions exist.

Insofar as insulin sensitivity may be increased by GPA, and therefore cell membrane permeability may be increased as well, those compounds that appear to increase protein synthesis (perhaps by affecting a positive nitrogen balance) such as Mono- or DiCreatine-HMB, Amino Acids like Alanine, and L-Glutamine, may be more efficiently transported into the cell in the presence of GPA. Additionally, other beneficial effects of insulin, such as its anabolic and anticatabolic actions, its positive effect on protein, triglyceride, and HDL formation by the liver, and its stimulation of ribosomal protein synthesis, may be maximized as well.

The amount of GPA will typically be from about 10 mg to about 1000 mg daily. It should be noted that this may be, in certain embodiments, administered in various doses throughout the day.

### TMG

TMG appears to assist protein synthesis. TMG functions as a methyl donor, which increases methylation in the body, and so produces methionine, necessary for protein synthesis. Thus, the muscle growth process may be accelerated.

The amount of TMG will typically be from about 10 mg to about 10 grams daily. It should be noted that this may be, in certain embodiments, administered in various doses throughout the day.

It should be understood that any ratios set forth herein are merely a preferred implementation and in no way limit the composition to any ratio described above, as people skilled in the art will recognize that variations in any ratio between substances will not significantly alter the properties of the present invention.

It should be further understood that this invention, and the aspects thereof, allow for the substitution of any of the compounds with a pharmaceutical and biochemical equivalent or complement, as long as that substitute functions in the same manner as the compound which it replaces in the composition.

### Formulation

A representative formula for an preferred powder embodiment comprises:
5 grams by weight of Dicreatine-HMB salt;
500 mg by weight of Putrescine;
2 grams by weight of Alanine-Glutamine 1:1 molecular ratio dipeptide or free base compound;
1000 mg by weight of TMG; and,
500 mg of GPA.

Of course this formula is suitable to variation in both the amount of a compound or the substitution of one of the above compounds in the composition with an analogous compound of similar function either as is described in this application or as known in the art.

Dosages may be varied as desired. For example, a single dose of the representative compound described above may be administered; some compounds may be administered in a single dose, with others in another dose administered substantially concomitantly; compounds may be administered separately, etc. Dosage forms may be as desired as well, e.g. tablets, capsules, etc. with appropriate additions as necessary or desired (e.g. excipients in a sufficient quantity of each to make a suitable tablet, etc.)

### Administration

The dose can be given from 1 to about 6 times daily, preferably from 2 to about 3 times daily.

Compositions according to various embodiments may be formulated for administration by any suitable route such as the oral, rectal, nasal, topical (dermal) or parenteral administration route, and be in the form of tablets, capsules, suspensions, emulsions, solutions, injectables, suppositories, sprays, aerosols, sustained release compositions and/or devices, or others as desired.

Formulations for oral use include tablets which contain the active ingredient in admixture with non-toxic pharmaceutically acceptable excipients, as known in the art, for example, inert diluents, e.g. calcium carbonate, sodium chloride, lactose, calcium phosphate or sodium phosphate; granulating and disintegrating agents, e.g. potato starch or alginic acid; binding agents, e.g. starch, gelatin or acacia; lubricating agents, e.g., magnesium stearate, stearic acid or talc, etc.

Other pharmaceutically acceptable excipients can be colorants, flavouring agents, plasticizers, humectants etc. The tablets may be uncoated or they may be coated by known techniques, optionally to delay disintegration and absorption in the gastrointestinal tract and thereby provide a sustained action over a longer period. For example, a time delay material such as glyceryl monostearate or glyceryl distearate may be employed.

Formulations for oral use may also be presented as chewing tablets, or as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin, or olive oil.

Powders, dispersible powders or granules suitable for preparation of an aqueous suspension by addition of water may also be used as convenient dosage forms. Formulation as a suspension provide the active ingredient in admixture with a dispersing or wetting agent, suspending agent and one or more preservatives. Suitable dispersing or wetting agents are, for example, naturally-occurring phosphatides, as e.g. lecithin, or condensation products of ethylene oxide with e.g. a fatty acid, a long chain aliphatic alcohol or a partial ester derived from fatty acids and a hexitol or a hexitol anhydrides, for example, polyoxyethylene stearate, polyoxyethylene sorbitol monooleate, polyoxyethylene sorbitan monooleate etc. Suitable suspending agents are, for example, sodium carboxymethylcellulose, methylcellulose, sodium alginate etc.

The pharmaceutical formulation may also be administered parenterally (intravenous, intramuscular, subcutaneous or the like) in dosage forms or formulations containing conventional, non-toxic pharmaceutically acceptable carriers and adjuvants, as known in the art.

Formulations for rectal application include suppositories (emulsion or suspension type), and rectal gelatin capsules (solutions or suspensions). Appropriate pharmaceutically acceptable suppository bases are used, such as cocoa butter, esterified fatty acids, glycerinated gelatin, and various water-soluble or dispersible bases like polyethylene glycols and polyoxyethylene sorbitan fatty acid esters. Various additives like e.g. enhancers or surfactants may be incorporated.

Formulations for nasal application include nasal sprays and aerosols for inhalation. In a typically nasal formulation, the active ingredients are dissolved or dispersed in a suitable vehicle. The pharmaceutically acceptable vehicles and excipients and optionally other pharmaceutically acceptable materials such as diluents, enhances, flavouring agents, preservatives etc. are all selected in accordance with conventional pharmaceutical practice in a manner understood by the persons skilled in the art of formulating pharmaceuticals.

Formulations for topical application for percutaneous absorption in dosage forms or formulations contain conventionally non-toxic pharmaceutically acceptable carriers and excipients including microspheres and liposomes as known in the art. The formulations include creams, ointments, lotions, liniments, gels, hydrogels, solutions, suspensions, pastes, plasters and other kinds of transdermal drug delivery systems. The pharmaceutically acceptable carriers or excipients may include emulsifying agents, antioxidants, buffering agents, preservatives, humectants, penetration enhancers, chelating agents, gelforming agents, ointment bases, perfumes and skin protective agents. Examples of emulsifying agents are naturally occurring gums, e.g. gum acacia or gum tragacanth, naturally occurring phosphatides, e.g. soybean lecithin and sorbitan monooleate derivatives. Examples of antioxidants are butylated hydroxy anisole (BHA), ascorbic acid and derivatives thereof, tocopherol and derivatives thereof and cysteine. Examples of preservatives are parabens and benzalkonium chloride. Examples of humectants are glycerin, propylene glycol, sorbitol and urea. Examples of penetration enhancers are propylene glycol, DMSO, triethanoiamine, N,N-dimethylacetamide, N,N-dimethylformamide, 2-pyrrolidone and derivatives thereof, tetrahydrofurfuryl alcohol and Azone. Examples of chelating agents are sodium EDTA, citric acid and phosporic acid. Examples of gel forming agents are Carbopol, cellulose derivatives, bentonit, alginates, gelatin and PVP. Examples of ointment bases are beeswax, paraffin, cetyl palmitate, vegetable oil, sorbitan esters of fatty acids (Span), polyethyleneglycols, and condensation products between sorbitan esters of fatty acids and ethylene oxide, e.g. polyoxyethylene sorbitan monooleate.

It should be understood that the ratios set forth herein are merely a preferred implementation and in no way limit the composition to the ratio described above, as people skilled in the art will recognize that variations in the ratio between substances will not significantly alter the properties of the present invention. It should be further understood that said ratios and/or amounts used may be adjusted for weight, and/or other predetermined factors. So, for example, dosages may be adjusted for an individual who is exercising heavily and readjusted for an individual whop is in a passive or resting exercise phase.

It should be further understood that this invention, and the aspects thereof, allow for the substitution with a pharmaceutical and biochemical equivalent or complement, as long as that substitute functions in the same manner as the compound which it replaces in the composition.

1) A method of providing nutrient compositions comprising:
providing various nutrients for increasing bodily energy balance, which comprises:
providing at least one nutrient in order to assist in fat oxidation and so provide glucose for energy;
providing at least one nutrient in order to assist in gluconeogenesis and so provide glucose for energy;
providing at least one nutrient in order to assist in conversion of existing muscular energy stores;
providing at least one nutrient in order to assist in transport of any of above said nutrients into a muscle cell;
providing various nutrients for decreasing muscle catabolism; and,
providing various nutrients for increasing protein synthesis.

2) A method as in claim 1 wherein:
said providing at least one nutrient in order to assist in fat oxidation and so provide glucose for energy further comprises providing HMB;
said providing at least one nutrient in order to assist in gluconeogenesis and so provide glucose for energy further comprises providing L-Alanine;
said providing at least one nutrient in order to assist in conversion of existing muscular energy stores further comprises providing Creatine;
said providing at least one nutrient in order to assist in transport of any of above said nutrients into a muscle cell further comprises providing GPA;
said providing various nutrients for decreasing muscle catabolism further comprises providing Glutamine; and,
said providing various nutrients for increasing protein synthesis further comprise providing Putrescine and TMG.

3) A method according to claim 2, wherein the administering step is performed on a daily basis.

4) A method according to claim 2, wherein the administering step is performed following an exercise period.

5) A method of providing nutrient compositions comprising providing energy through exogenous nutrients, which further comprises providing a first compound that may utilize endogenous fat stores; providing a second compound that may increase transport of endogenous energy stores; providing a third compound that may increase available energy to a muscle; providing a fourth compound that may assist in anticatabolic reactions and providing a fifth compound that may increase protein synthesis.

6) An composition for enteral or parenteral administration comprising:
Mono- or Dicreatine-HMB salt;
Putrescine Dihydrochloride;
Alanine
L-Glutamine;
Trimethylglycine; and,
Guanidinopropionic Acid.

7) A composition according to claim 6 wherein the molecular ratio of Alanine to L-Glutamine ranges from 1:2 to 2:1.

8) An composition for enteral or parenteral administration comprising:
1 to 10 grams by weight of Mono- or Dicreatine-HMB salt;
10 mg to 10 grams by weight of Putrescine Dihydrochloride;
1 to 30 grams by weight of an amino acid compound comprising Alanine chemically bound at a 1:1, 2:1 or 1:2 molecular ratio to L-Glutamine;
100 mg to 10 grams by weight of Trimethylglycine; and,
10 mg to 5 grams of Guanidinopropionic Acid.

9) The composition of claim 8 formulated for enteral administration comprising said unit dosage form admixed with flavors and sweeteners.

10) The composition of claim 8 formulated for enteral administration comprising said unit dosage form contained in blended powder or one or more capsules.

11) A composition according to claim 8 wherein said composition is in the form of a powder, tablet, capsule, pill, liquid, food additive, candy, confection or nutrition bar.

12) A composition according to claim 11 wherein said powder is admixed with a liquid.

13) The composition of claim 8 in a sustained release form.

14) A method for enhancing the physical endurance of a mammal by administering to said mammal a therapeutically effective amount a composition comprising of:
Mono- or Dicreatine-HMB salt;
Putrescine Dihydrochloride;
Alanine
L-Glutamine;
Trimethylglycine; and,
Guanidinopropionic Acid.

15) A method for increasing the energy balance in a mammal which comprises administering to a mammal a therapeutically effective amount of a composition comprising:
Mono- or Dicreatine-HMB salt;
Putrescine Dihydrochloride;
Alanine
L-Glutamine;
Trimethylglycine; and,
Guanidinopropionic Acid.

16) A method according to claim 14, wherein administering is performed on a daily basis.

17) A method according to claim 14, wherein administering is performed following an exercise period.

18) A method according to claim 14, wherein the composition comprises an amount of from about 5 grams to about 100 grams per day.

19) A method according to claim 14, wherein the composition comprises an amount of about 9 grams to about 27 grams per day.

20) An composition comprising 50% Dicreatine-HMB; 20% Alanyl-L-Glutamine; 10% TMG; 5% Putrescine Dihydrochloride; 5% GPA and 10% flavors and sweeteners.

21) A method according to claim 16, wherein said daily administration comprises at least two partial daily administrations of said composition.

22) A composition according to claim 8 wherein said compound weights are adjusted according to predetermined factors.

23) A composition according to claim 22 wherein said predetermined factors include an individual's weight.

24) A composition according to claim 22 wherein said predetermined factors include an individual's exercise intensity.

25) A composition according to claim 22 wherein said predetermined factors include an individual's lean body mass.

26) A composition according to claim 22 wherein said predetermined factors include an individual's proportion of body fat to lean body mass.

27) A composition according to claim 22 wherein said predetermined factors include an individual's progress along a loading cycle.

28) A method according to claim 1, wherein administering is performed on a daily basis.

29) A method according to claim 28, wherein said daily administration comprises at least two partial daily administrations of said composition.
